(19) United States
(12) Patent Application Publication
(10) Pub. No.: US 2009/0099664 A1
(43) Pub. Date: Apr. 16, 2009

(54) SUBTALAR IMPLANT AND KIT

(76) Inventor: Perry C. Forrester, Houston, TX (US)

Correspondence Address: C. JAMES BUSHMAN, 5851 San Felipe, SUITE 975, HOUSTON, TX, 77057, US

(21) Appl. No.: 12/251,863

(22) Filed: Oct. 15, 2008

Related U.S. Application Data
(60) Provisional application No. 60/999,094, filed on Oct. 16, 2007.

(51) Int. Cl. A61B 17/56; A61F 2/42

(52) U.S. Cl. 623/21.18; 606/86 R

(57) ABSTRACT

A subtalar implant comprising a body with a proximal end and a distal end, the body including a cylindrical portion proximate the proximal end, an externally threaded frustoconical portion extending generally from the cylindrical portion toward the distal end, an axially extending bore through the proximal end and the distal end, a driver formation coaxial with the bore and a female threaded portion coaxial with the bore and extending from about the proximal end toward the driver formation, the threads on the externally threaded portion being of an opposite turn to the threads on the male threaded portion.

### CROSS REFERENCE TO RELATED APPLICATION

This application claims the priority of U.S. Provisional Application No. 60/999,094 filed on Oct. 16, 2007, the disclosure of which is incorporated herein by reference for all purposes.

### BACKGROUND OF THE INVENTION

1. Field of the Invention

The present invention relates to a surgical implant more specifically a subtalar implant to correct podiatric disorders and a bit useful in performing surgical procedure.

2. Description of Prior Art

Subtalar implants for correcting podiatric disorders including flat foot dates back to the 1940's. Originally, to treat flat foot a bone graft or silicone wedge was used to elevate the sinus tarsi and to limit pronation of the subtalar joint. These techniques gave way to the use of a stemmed polyethylene block. All of the above methods were later replaced with modern, threaded implants for surgical implantation within the sinus tarsi. This is exemplified, for example, in U.S. Pat. No. 6,168,631 to Maxwell et al. which discloses a subtalar implant comprised of a cylindrical body which is sized to fit within the sinus tarsi of a subtalar joint, the body having at least one external, longitudinally formed slot, the slot being sized and shaped to render the implant sufficiently resilient to dissipate forces upon the implant in normal use by the patient. The Maxwell implant includes an engagement element to engage an insertion tool to enable rotation of the implant about its longitudinal axis and thus thread the implant into place in the sinus tarsi.

Generally speaking modern implants are either cylindrical or conical in shape, the cylindrically shaped implants generally providing a less desirable anatomical fit than the conically shaped implants. However, the latter require apertures along the external thread surface for post implantation tissue growth to stabilize the implant.

### SUMMARY OF THE INVENTION

The present invention, in one embodiment, provides a subtalar implant capable of threaded engagement in the sinus tarsi using a insertion tool for manipulating the implant during the surgical implantation, the implant also including an internal structure which allows the use of a removal tool to remove or reposition the implant if and when necessary.

In one embodiment, the subtalar implant of the present invention comprises an elongate body having a conical portion and a cylindrical portion, at least a portion of the conical portion being provided with external or male threads. The body of the implant of the present invention has a proximal end and a distal end, a bore extending axially through the body of the implant, the cylindrical portion being approximately adjacent the proximal end. Formed in the body near the proximal end of the implant is an internally threaded section, i.e., female threads, which have the opposite turn of the external threads, i.e., if the external thread is a right-hand thread, the internal thread is a left-hand thread. Axially inward of the internally threaded section is a driver formation, e.g., a socket, for engagement by a driver to drive the implant into the subtalar joint.

In another embodiment, the present invention provides a kit useful in certain surgical procedures such as correcting podiatric disorders as, for example, using subtalar implants. The kit of the present invention can comprise a tray having a plurality of first and second receiving formations. Selectively received in each of the first receiving formations are respective ones of a series of sizing tools. Selectively received in each of the second receiving formations are respective ones of a series of different sized implants. At least a portion of each of the sizing tools is formed of titanium, the titanium portion of respective ones of said sizing tools being anodized with a distinct color. The implants are made of titanium, respective ones of the implants being anodized with a distinct color. The anodized colors of the respective ones of the sizing tools are matched with the anodized colors of respective ones of the implants.

The kit can also include a driver tool, a removal tool and a locating probe for locating a body aperture, e.g., the sinus tarsi.

### BRIEF DESCRIPTION OF THE DRAWINGS

FIG. 1 is a side, elevational view of a subtalar implant of the present invention.

FIG. 2 is a cross-sectional view taken along the lines 2-2 of FIG. 1.

FIG. 3 is a cross-sectional view along the lines 3-3 of FIG. 1.

FIG. 4 is a cross-sectional view taken along the lines 4-4 of FIG. 1.

FIG. 5 is an end view of the implant of FIG. 1.

FIG. 6 is a side, elevational view of a driver tool used with the implant of the present invention.

FIG. 7 is an end view of the driver tool shown in FIG. 6.

FIG. 8 is a side, elevational view of a removal tool used to remove the implant of FIG. 1 from the sinus tarsi, and FIG. 9 is a top plan view of a kit incorporating the implants of the present invention.

### DESCRIPTION OF THE PREFERRED EMBODIMENTS

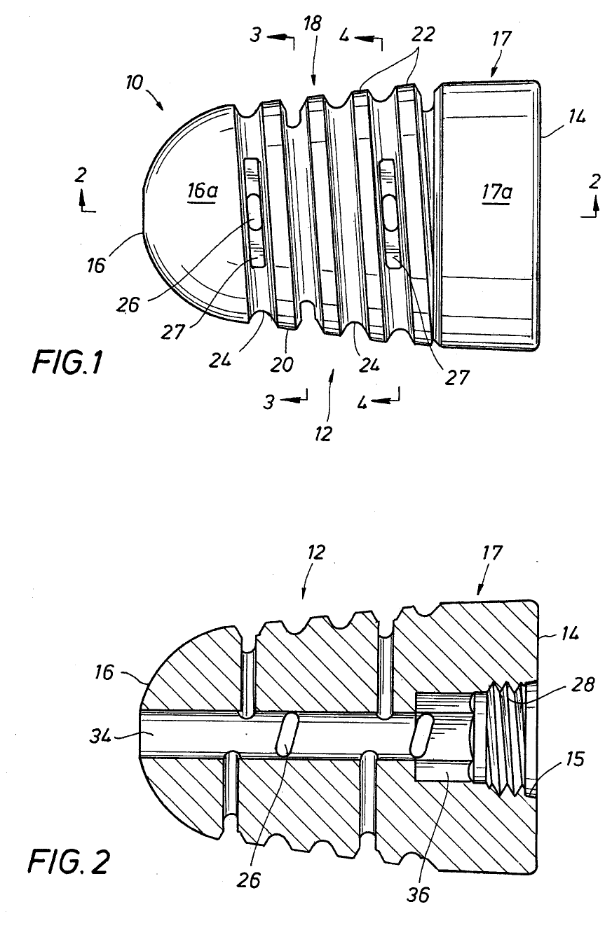

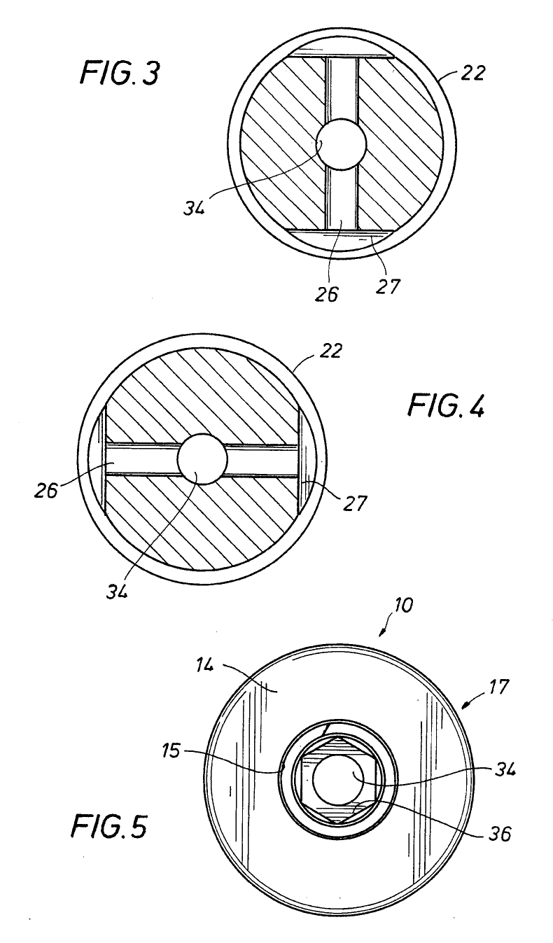

Referring first to FIG. 1, the implant, shown generally as 10, includes an elongate body 12 having a proximal end with a planar face 14 and a distal end 16 having a domed shape. As can be seen from FIG. 1, body 12 has a cylindrical portion 17 having a cylindrical surface 17a proximate the planar face 14 of the proximal end and a conical portion 18, conical portion 18 extending axially from the cylindrical portion 17 toward the domed shape portion 16a on the distal end 16 and being provided with tapered male threads 20 having frustoconical crests 22 and radiused roots 24. As can be seen with reference to FIGS. 1 and 2, apertures 26 are formed in grooves 27 in the roots 24 and extend through the body 12 and are in open communication with a bore 34 which extends from and through proximal end 14 to and through distal end 16. Grooves 27 and apertures 26 provide receptacles for post implantation osseous tissue growth to further stabilize the implant 10.

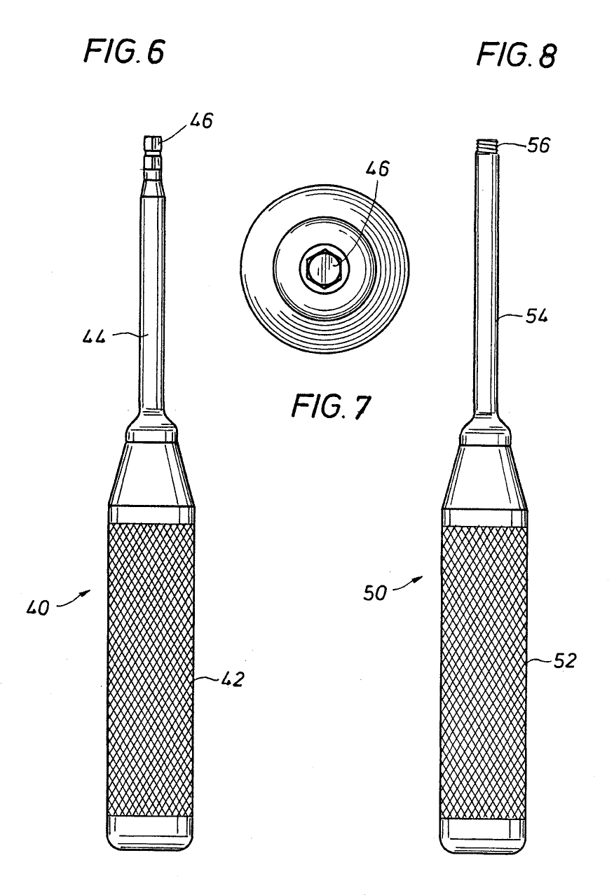

Bore 34 has a small, chamfered (frustoconical) surface 15 in open communication with tapered female threads 28 which, as seen, are coaxial with bore 34 and extend generally from about proximal end 14, i.e., from chamfered surface 15, axially inward along bore 34. Displaced axially, inwardly from threads 28 is a driver engagement formation 36 in open communication and coaxial with bore 34. Formation 36 is adapted to be engaged by a driver tool (discussed hereafter), engagement formation 36 being of a formation which is generally complimentary to the drive head of the driver tool. While as shown engagement formation 36 is hexagonal when viewed in plan view, it could be of numerous other types of formations as, for example, a recess having a cruciform, rectangular, octagonal, or other shapes. Alternatively, formation 36 could be provided with projecting formations which would be received in the drive head of the driver tool 40 (FIG. 6).

Referring now to FIG. 6, a driver used with implant 10 is shown. The driver shown generally as 40 comprises a knurled handle portion 42 attached to a rigid shank 44. The end of shank 44 distal handle portion 42 terminates in a driver head 46 for engaging engagement formation 36. By example, if formation 36 is a hexagonal socket, as shown, head 46 would be hexagonal in shape and size. Accordingly, as will be understood by those skilled in the art, when head 46 of driver 40 is engaged in engagement formation 36, the implant 10 can be rotated and, assuming thread 20 is a right-hand thread, would be rotated clockwise to drive implant 10 into the sinus tarsi.

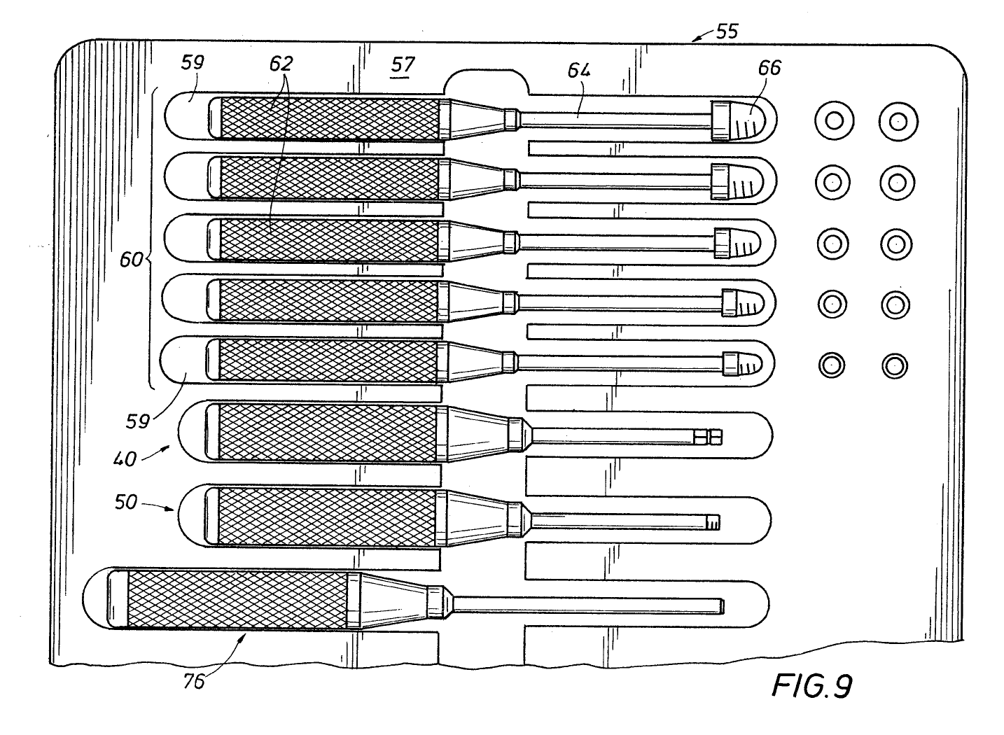

As noted above, the implant 10 of the present invention can be easily removed from and/or repositioned in the sinus tarsi if and when necessary. To this end, and as discussed above, threads 28 are of the opposite turn from threads 20, i.e., if thread 20 is a right-hand thread, thread 28 is a left-hand thread and vice versa. In any event, assuming thread 20 is a right-hand thread and thread 28 is a left-hand thread, to remove implant 10 from the sinus tarsi, a removal tool shown generally as 50 in FIG. 8 is employed. Removal tool 50 has a knurled handle 52, a rigid shank 54, and a threaded head 56 which, in the example just given, is a left-hand thread and is complimentary to threads 28. Accordingly, when threaded head 56 is fully engaged in thread 28, counterclockwise rotation of tool 50 will back implant 10 out of the sinus tarsi.

The kit of the present invention, shown generally as 55 in FIG. 9 includes a tray 57 having a first set of recesses 59 and a second set of recesses (not shown) but described hereafter. Respective ones of a series of sizer probes, shown generally as 60, are received in respective ones of the first receiving formations 59, each receiving formation 59 receiving a different sized probe 60. Probe 60 comprise a knurled handle portion 62, a shank 64 and a sizer head 66. The knurled portion of handle portions 62 of the various sized, sizer probes, are made of titanium and they are anodized with a distinct color. Thus, a sizer probe for a 8 mm implant could be dark blue, a sizer probe for a 9 mm implant could be green, etc.

As noted, there are a second set of recesses in each of which is received a implant 10. As shown, there are dual sets of the second receiving formations such that two implants 10 of each size can be included in the kit 55. Thus, in one pair of the second recesses 10 mm implants 10 can be disposed, and in another pair of the second recesses, 9 mm implants can be disposed, etc. The implants 10 are made of titanium and each same sized pair of the implants 10 comprise a distinct anodized color, the anodized color on a given size of implants substantially exactly matching the anodized color on the appropriate sizer probe received in the recesses 59. Thus, and by example only, an 8 mm implant would have the same blue color as the sizer 60 for the 8 mm implant, the 9 mm implant would have the same color as the color of the sizer for the 9 mm implant, etc. As noted, the portion of the sizer probe, e.g., the knurled handle which is anodized with a distinct color is made of titanium and the implants 10 are likewise made of titanium. The use of titanium on the implants 10 and the knurled handle portions 62 of the sizer probes 60 ensures virtually perfect color matching. In other words, a given size probe 60 having a handle portion 62 with a given anodized color can be quickly and accurately matched to the right sized implant 10 because of the fact that the color on the size of the implant 10 and the color on the matching size probe 60 are for all intents and purposes indistinguishable. Thus, the surgeon can quickly and accurately select a correctly sized implant 10 to provide the desired degree of stabilization when the implant 10 is driven into the sinus tarsi. As also shown in FIG. 9, in addition to the sizer probes 60 and the implants 10, the kit can also include a locating probe 76, the driver 40 described above and the removal tool 50 described above.

Obviously the kit 55 can be comprised of any number of sizer probes 60 and matching size implants 10 and can in addition to the other tools mentioned, include additional tools or devices depending upon the particular surgical procedure involved.

The implant 10 of the present invention has several distinct advantages when compared with prior art subtalar implants. For one, the relatively large, smooth cylindrical surface 17a of the cylindrical portion 17 minimizes sinus tarsitis which frequently occurs when an implant is threaded its entire length. Additionally, the relatively large cylindrical surface 17a provides an enhanced support area to stabilize the bone structure when the implant 10 is in place. Additionally, the cylindrical surface 17a provides a large bearing area for distributing forces generated as the user walks or otherwise manipulates the foot thereby enhancing the healing process.

The implant 10 of the present invention also has advantages in the relative positioning of the engagement or driver formation 36 and the female threads 28 which can be referred to as removal threads as described above when it is desired to remove the implant 10 from the foot. Because the removal threads 28 extend virtually from the planar face 14, they are much easier to locate if removal is desired. As is well known, over time after the implant 10 is in place, tissue including scar tissue as well as penetrating the holes 26 in the grooves 27 can also cover the planar surface 14 of the proximal end of the implant 10 making it difficult, when removal is desired to easily locate the removal threads 28. If the axial positioning of the removal threads 28 and the engagement formation 36 were reversed, locating the removal threads 28 would be more difficult since a much larger volume of scar tissue would have to be penetrated to reach the removal threads. This will result in greater trauma to the tissue and hinder the healing process. Furthermore, by locating the removal threads 28 near the planar face 14, the chamfered surface 15 acts as a guide, e.g., a funnel, to guide the threaded removal head 46 of removal tool 50 into the removal threads 28.

The foregoing description and examples illustrate selected embodiments of the present invention. In light thereof, variations and modifications will be suggested to one skilled in the art, all of which are in the spirit and purview of this invention.

What is claimed is:

1. A subtalar implant comprising:
a body having a proximal end and a distal end, said body including a cylindrical portion proximate said proximal end, an externally threaded frustoconical portion extending from said cylindrical portion toward said distal end, an axially extending bore through said proximal end and said distal end, a driver formation coaxial with said bore, a female threaded portion coaxial with said bore and extending from about said proximal end toward said driver formation, the threads on said externally threaded portion being of an opposite turn to threads on said male threaded portion.

2. The implant of claim 1 when said distal end is a domed shape.

3. The implant of claim 1 wherein the crests of said male threads are frustoconical.

4. The implant of claim 3 wherein the roots of said male threads are radiused.

5. The implant of claim 1 wherein there are apertures in the roots of said male threads in open communication with said bore.

6. The implant of claim 1 wherein there is a chamfered surface extending from a planar surface on said proximal end to said female threaded portion.

7. A kit for use in conducting surgical procedures comprising:
a tray having a plurality of first receiving formations and a plurality of second receiving formations;
a series of sizing tools, respective ones of said sizing tools being selectively received in respective ones of said first receiving formations, at least a portion of each of said sizing tools being made of titanium, each of said titanium portions having a distinct anodized color;
a series of surgical implants, respective ones of said surgical implants being selectively received in respective ones of said second receiving formations, each of said surgical implants being made of titanium and each having a distinct anodized color, the anodized colors on respective ones of said sizing tools substantially exactly matching the anodized colors on respective ones of said implants.

8. The kit of claim 7, wherein said tray includes a driver receiving formation, a retriever tool formation and a locating probe formation.

9. The kit of claim 7, wherein said surgical implants comprise subtalar implants.

10. The kit of claim 7, wherein said sizing tools comprise a handle portion, a shank portion and a sizer head extending from said shank portion.
